(19) United States
(12) Patent Application Publication
(10) Pub. No.: US 2025/0152570 A1
(43) Pub. Date: May 15, 2025

(54) Uses of Selonsertib in the Preparation of Drugs for the Treatment of Cancer

(71) Applicant: AIR FORCE MEDICAL UNIVERSITY, Xi'an (CN)

(72) Inventors: Zhe Wang, Xi'an (CN); Mingyang Li, Xi'an (CN); Jia Chai, Xi'an (CN); Yanru Yang, Xi'an (CN); Jing Ma, Xi'an (CN); Linni Fan, Xi'an (CN); Yixiong Liu, Xi'an (CN); Yao Lv, Xi'an (CN); Junpeng Xu, Xi'an (CN); Tianqi Xu, Xi'an (CN); Shoubin Qian, Xi'an (CN); Shuai He, Xi'an (CN); Jin Liu, Xi'an (CN); Junyang Song, Xi'an (CN); Lingfei Li, Xi'an (CN)

(73) Assignee: AIR FORCE MEDICAL UNIVERSITY, Xi'an (CN)

(21) Appl. No.: 19/019,396

(22) Filed: Jan. 13, 2025

Related U.S. Application Data
(63) Continuation of application No. PCT/CN2023/138288, filed on Dec. 13, 2023.

(30) Foreign Application Priority Data
Dec. 28, 2022 (CN) CN202211692715.9

(51) Int. Cl. A61K 31/4439; A61K 39/395; A61P 35/00

(52) U.S. Cl. CPC A61K 31/4439; A61K 39/3955; A61P 35/00

(57) ABSTRACT

The present invention belongs to the field of medicine and specifically relates to the use of Selonsertib in the preparation of drugs for the treatment of cancer. The Selonsertib is the English name of the compound 5-(4-cyclopropyl-1H-imidazole-1-yl)-2-fluoro-N-(6-(4-isopropyl-4H-1,2,4-triazol-3-yl)pyridine-2-yl)-4-methylbenzamide, and its chemical formula is C24H24FN7O, CAS No.: 1448428-04-3. The results of the present invention show that Selonsertib can efficiently inhibit the activity of RSK4 kinase, and has a significant inhibitory effect on the growth of a variety of tumors, and can reduce the resistance of tumors to EGFR-TKI-targeted drugs when combined with EGFR-TKI-targeted drugs. Selonsertib in combination with Anti-PD1 has a significant effect on inhibiting tumor growth compared with the drug alone. The pharmaceutical composition containing Selonsertib provided by the present invention has a good application prospect in the targeted therapy of a variety of cancers.

### TECHNICAL FIELD

The present invention belongs to the field of medicine and specifically relates to the use of Selonsertib in the preparation of drugs for the treatment of cancer.

### BACKGROUND TECHNOLOGY

RSK4 (ribosomal s6 kinase 4, RSK4), also known as RPS6KA6, is a gene obtained by Yntema et al. when they studied patients with X-chromosome-linked intellectual disability, which is located at the site of X chromosome q21 and can encode a protein of about 746 amino acids It belongs to the RSK kinase family and is a silk/threonine protein kinase. To date, four RSK subtypes (RSK-1, RSK-2, RSK-3, and RSK-4) have been identified, which share the same basic structure but completely different functions.

RSK4 is a protein kinase that some inventors of the present invention have been concerned about. RSK4 may be a cancer-promoting factor with high expression in esophageal squamous cell carcinoma, renal cell carcinoma, and pancreatic cancer, and is associated with poor prognosis in patients. The inventor of the present application found that RSK4 is highly expressed in esophageal squamous cell carcinoma (ESCC) and kidney cancer (RCC) and promotes the invasion and metastasis of tumor cells, and plays an important role in promoting radiotherapy resistance and enhancing tumor stemness in ESCC. It is of great clinical significance to explore small molecule kinase inhibitors specifically targeting RSK4 and verify their anti-tumor activity in cancer, in order to expand and make up for the types and shortcomings of existing RSK4 inhibitors, and to provide potential therapeutic targets and theoretical basis for small molecule targeted therapy for cancer.

In recent years, with the rapid development of tumor immunology, major breakthroughs have been made in tumor immunotherapy, and T-cell immune checkpoint inhibitors are the main research directions of tumor immunotherapy, the most representative of which are PD-1/PD-L1 inhibitors. Monoclonal antibody drugs targeting PD-1 and its ligand PD-L1 block PD1/PD-L1 negative immune examination sites to activate and proliferate T-cells, which can reverse the tumor immunosuppressive microenvironment, enhance the anti-tumor immune response, and effectively inhibit the growth of tumors. In the treatment of cancer, immunotherapy still cannot achieve effective treatment for most cancer patients, and the sample size of existing clinical studies is limited, and clinical research data still need to be further accumulated. The therapeutic effect of separate administration has certain limitations, the drug effect is not obvious, and it is easy to develop drug resistance. With the advancement of cancer treatment, emerging treatment options, such as new chemotherapy drugs, triple therapy, immunotherapy and small molecule inhibitor therapy, are also being gradually applied to the treatment of cancer to improve its poor prognosis.

### CONTENTS OF THE INVENTION

In order to solve the above technical problems, the present invention provides the use of compound 5-(4-cyclopropyl-1H-imidazol-1-yl)-2-fluoro-N-(6-(4-isopropyl-4H-1,2,4-triazol-3-yl)pyridine-2-yl)-4-methylbenzamide (Selonsertib) in the preparation of drugs for the treatment of cancer, the cancer comprises esophageal squamous cell carcinoma, Head and neck squamous cell carcinoma, pancreatic cancer, prostate cancer, urothelial cancer, lung cancer and melanoma, the chemical formula of the compound 5-(4-cyclopropyl-1H-imidazol-1-yl)-2-fluoro-N-(6-(4-isopropyl-4H-1,2,4-triazol-3-yl)pyridine-2-yl)-4-methylbenzamide is C24H24FN7O, and the structural formula is as follows:

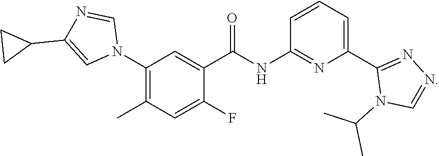 [SMILES: Cc1cc(F)c(C(=O)Nc2cccc(-c3nncn3C(C)C)n2)cc1-n1cnc(C2CC2)c1]

Further, said cancers include esophageal squamous cell carcinoma, head and neck squamous cell carcinoma, pancreatic cancer, prostate cancer, urothelial carcinoma, lung cancer, or melanoma.

Further, the compound 5-(4-cyclopropyl-1H-imidazol-1-yl)-2-fluoro-N-(6-(4-isopropyl-4H-1,2,4-triazol-3-yl)pyridin-2-yl)-4-methylbenzamide combined with EGFR-TKI targeted drug is used to prepare drugs for the treatment of cancer, and the EGFR-TKI is an epidermal growth factor receptor tyrosine kinase inhibitor.

Further, the EGFR-TKI-targeted drug and compound 5-(4-cyclopropyl-1H-imidazol-1-yl)-2-fluoro-N-(6-(4-isopropyl-4H-1,2,4-triazol-3-yl)pyridine-2-yl)-4-methylbenzamide are administered continuously at the same time or in any sequence.

Further, said cancers include esophageal squamous cell carcinoma, head and neck squamous cell carcinoma, pancreatic cancer, prostate cancer, urothelial cancer, lung cancer, and melanoma.

Further, the drug is used for the treatment of esophageal squamous cell carcinoma, head and neck squamous cell carcinoma, pancreatic cancer, prostate cancer, urothelial carcinoma, lung cancer and melanoma that have developed resistance to EGFR-TKI-targeted drugs, or for the prevention and treatment of esophageal squamous cell carcinoma, head and neck squamous cell carcinoma, pancreatic cancer, prostate cancer, urothelial carcinoma, lung cancer and melanoma acquired resistance to EGFR-TKI-targeted drugs, or for the prevention and treatment of esophageal squamous cell carcinoma, head and neck squamous cell carcinoma, pancreatic cancer, prostate cancer, Urothelial carcinoma, lung cancer, and melanoma recur after discontinuation of treatment with EGFR-TKI-targeted drugs.

Further, the EGFR-TKI targeted drugs include first-generation drugs: icotinib, gefitinib, erlotinib, second-generation drugs: afatinib, and third-generation drugs: osimertinib.

Further, the compound 5-(4-cyclopropyl-1H-imidazol-1-yl)-2-fluoro-N-(6-(4-isopropyl-4H-1,2,4-triazol-3-yl)pyridine-2-yl)-4-methylbenzamide combined with Anti-PD1 is used to prepare drugs for the treatment of cancer, and the Anti-PD1 is a humanization IgG4 antibody is an inhibitor of PD-1, the PD-1 is programmed death receptor 1, and Anti-PD-1 includes: pembrolizumab, nivolumab, sintilimab, toripalimab, camrelizumab.

Further, the Anti-PD-1 and the compound 5-(4-cyclopropyl-1H-imidazol-1-yl)-2-fluoro-N-(6-(4-isopropyl-4H-1,2,4-triazol-3-yl)pyridine-2-yl)-4-methylbenzamide are administered simultaneously or in any sequence continuously.

Further, said cancers include esophageal squamous cell carcinoma, head and neck squamous cell carcinoma, pancreatic cancer, prostate cancer, urothelial cancer, lung cancer, and melanoma.

Compared with the prior art, the present invention has the following beneficial effects:

The present invention provides a new use of the compound 5-(4-cyclopropyl-1H-imidazol-1-yl)-2-fluoro-N-(6-(4-isopropyl-4H-1,2,4-triazol-3-yl)pyridine-2-yl)-4-methylbenzamide (Selonsertib), the results of the present invention show that Selonsertib can efficiently inhibit the expression of RSK4 enzyme, and has a positive effect on a variety of cancers (esophageal squamous cell carcinoma, Head and neck squamous cell carcinoma, pancreatic cancer, prostate cancer, urothelial cancer, lung cancer and melanoma) all have significant therapeutic effects, and when combined with EGFR-TKI-targeted drugs, they can reduce the resistance of tumors to EGFR-TKI-targeted drugs. In combination with Anti-PD-1, it can significantly improve the therapeutic effect of Anti-PD-1 on cancer.

### DESCRIPTION OF THE DRAWINGS

FIG. 1 shows the chemical structure of Selonsertib.

FIG. 2 shows the inhibition of the activity of RSK4 kinase by Selonsertib (IC50=50.44 nM).

FIG. 3 shows the effect of different concentrations of Selonsertib on the phosphorylation level of substrate downstream of RSK4 by Western blot. A: TE10 cells B: ECA109 cells.

FIG. 4 shows the effect of different concentrations of selonsertib on the proliferation of esophageal squamous cell carcinoma cells (TE10) (A) and the semi-inhibitory curve of selonsertib (B).

FIG. 5 shows the effect of different concentrations of selonsertib on the proliferation of esophageal squamous cell carcinoma cells (ECA109) (A) and the semi-inhibitory curve of selonsertib (B).

FIG. 6 shows the effect of different concentrations of selonsertib on the migration of esophageal squamous cell carcinoma cells (TE10) (A) and the semi-inhibitory curve of selonsertib (B).

FIG. 7 shows the effect of different concentrations of selonsertib on the migration of esophageal squamous cell carcinoma cells (ECA109) (A) and the semi-inhibitory curve of selonsertib (B).

FIG. 8 shows the inhibition of esophageal squamous cell carcinoma growth by Selonsertib in vivo, the treatment effect of each treatment group was compared in the tumorigenesis experiment of nude mice, and B is the tumor growth curve.

FIG. 9 shows that Selonsertib reverses the resistance of esophageal squamous cell carcinoma to icotinib. FIG. 10 shows that Selonsertib reverses the resistance of esophageal squamous cell carcinoma to afatinib.

FIG. 11 shows the inhibition of melanoma growth by Selonsertib enhanced by Anti-PD1, A: C57BL/6 mouse xenograft model to observe the treatment effect on different treatment groups, B, C: tumor growth curve and tumor weight comparison.

FIG. 12 shows the inhibition of Lewis lung cancer growth by Selonsertib enhanced by Anti-PD1, A: C57BL/6 mouse xenograft model to observe the treatment effect on different treatment groups, B, C: Tumor growth curve and tumor weight comparison.

### THE SPECIFIC EMBODIMENT

The present invention is described in detail below in conjunction with the accompanying drawings and specific embodiments, but should not be construed as a limitation of the present invention. If not otherwise specified, the technical means used in the following embodiments are conventional means well known to those skilled in the art, and the materials, reagents, etc. used in the following embodiments can be obtained from commercial means unless otherwise specified.

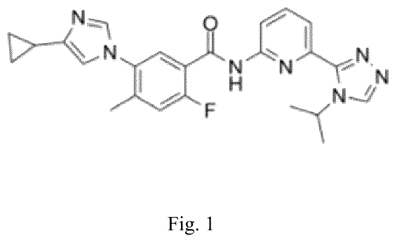

The present invention discloses a new use of the compound 5-(4-cyclopropyl-1H-imidazol-1-yl)-2-fluoro-N-(6-(4-isopropyl-4H-1,2,4-triazol-3-yl)pyridin-2-yl)-4-methylbenzamide (English name Selonsertib). The chemical formula of Selonsertib is C24H24FN7O, CAS No.: 1448428-04-3, and the structural formula is shown in FIG. 1.

The following Selonsertib, BI-D1870, Icotinib and afatinib were all purchased from Shanghai Taoshu Biotechnology Co., Ltd.

### Example 1: Inhibition of RSK4 Enzyme by Selonsertib

### 1 Experimental Methods

In this experiment, the ADP-Glo method was used to detect the effect of the compound to be tested on the RSK4 enzyme, and the starting concentration of the compound was 10 μM, 5-fold serial dilution, 2 replicates, and 6 concentrations.

(1) Thaw RSK4 enzyme, RSK Substrate, kinase assay buffer III (5× buffer), DTT (0.1M) and ATP (10 mM) on ice, and keep the above reagents on ice throughout the experiment.

(2) Prepare 5× buffer into 1× buffer with deionized water, and add DTT to it. The concentration of DTT in 1× buffer is 50 μM;

(3) Add 1 μl/well of 5× test compound to the white microplate, and centrifuge the microplate at 1000 rpm for 1 minute; Positive control well (Pos.Ctrl): 1 μl/well compound dilution solvent; Blank control well (Blank): 1 μl/well 1× buffer.

(4) After the RSK4 enzyme is completely thawed, dilute the RSK4 enzyme to 1 ng/μl with 1× buffer, take 2 μl/well and add it to the white microplate. At this time, the amount of RSK4 enzyme in each well is 2 ng; add 2 μl/well 1× buffer to the blank control well; this step should be performed on ice. After adding, centrifuge the microplate at 1000 rpm for 1 minute;

(5) Prepare RSK Substrate/ATP mixed solution: RSK Substrate/ATP mixed solution: take 130 μl of RSK Substrate (1 mg/ml), add 3.25 μl 5 mM ATP and 127 μl 2× buffer (note that this is diluted in proportion). At this time, the ATP concentration is 62.5 μM and the RSK Substrate concentration is 0.5 mg/ml; this step should be performed on ice;

(6) Take 2 μl/well of the RSK Substrate/ATP mixed solution to the white microplate. At this time, RSK The substrate concentration is 0.2 mg/ml, and the ATP concentration is 25 μM. After addition, the microplate is centrifuged at 1000 rpm for 1 minute;

(7) After centrifugation, apply a membrane to the microplate, press the membrane tightly, and incubate at 25° C. for 1 hour;

(8) Equilibrate the ADP-Glo™ reagent and Kinase Detection related reagents required in the Promega kit to room temperature, and mix the Kinase Detection buffer and Kinase Detection Substrate according to the instructions for use.

(9) After the incubation, take 5 μl/well ADP-Glo™ reagent and add it to the white microplate. Centrifuge the microplate at 1000 rpm for 1 minute and incubate at 25° C. for 40 minutes;

(10) After the incubation, take 10 μl/well Kinase Detection mixed solution and add it to the microplate. Centrifuge the microplate at 1000 rpm for 1 minute and incubate at 25° C. for 30 minutes;

(11) After the incubation, perform chemiluminescence detection on the plate reader and read the luminescence value (RLU);

(12) Calculation of enzyme e inhibition rate: % Inhibition=100−(RLU (Sample)−RLU (Blank))/(RLU (Pos.Ctrl)−RLU (Blank))×100%.

### 2 Experimental Results

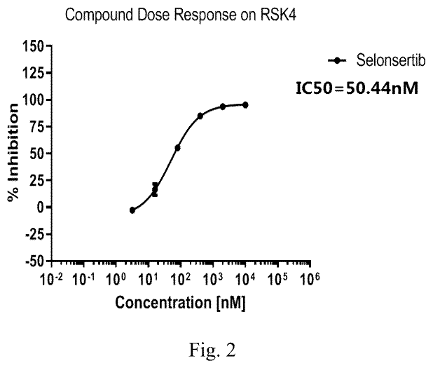

As shown in FIG. 2, with the increase of Selonsertib concentration, the inhibitory effect of Selonsertib on RSK4 kinase activity also increased. GraphPad software was used for curve fitting, and the IC50 value was 50.44 nM. The results showed that Selonsertib can effectively inhibit the phosphorylation of RSK4 and inhibit the activation of RSK4, and is an effective RSK4 inhibitor.

### Example 2: Effect of Selonsertib on the Phosphorylation Level of RSK4 Downstream Substrates

### 1 Experimental Method

In this experiment, Selonsertib was used to stimulate esophageal squamous cell carcinoma cell lines (TE10, ECA109) at different concentrations. Western blot was used to detect the effect of phosphorylation level of RSK4 downstream substrates.

(1) Protein extraction, using Kangwei Century RIPA lysis buffer. Place the cell culture flask on ice for 20 minutes, shake it back and forth, and blow it with a pipette. Scrape the cells with a cell scraper, transfer them to an EP tube, and centrifuge them at 4° C. 10-20 μl can be aspirated for protein quantification. Add loading buffer, boil for 5 min, centrifuge at 12000 rpm for 5 min, and store in an ultra-low temperature refrigerator. Tissue protein extraction: Grind in liquid nitrogen, transfer to EP tube after melting, lyse on ice for 20 min, and centrifuge at 4° C.

(2) Protein quantification, use the Kangwei Century BCA quantification kit. Make two replicate wells for each sample, take the average value to make a standard curve and calculate the sample concentration.

(3) Protein electrophoresis, use the Kangwei Century SDS-PAGE gel preparation kit. The separation gel concentration is 8%, and the concentration gel concentration is 5%. The separation gel is 1.5 cm away from the upper edge of the front glass plate and filled with water to seal the gel. Use filter paper to absorb the water sealing liquid, fill the concentrated gel, and insert the comb. The voltage is 100V constant for 1 h30 min until the bromophenol blue runs out and stops electrophoresis.

(4) Transfer to membrane, soak the gel, filter paper, and sponge pad in 4° C. pre-cooled transfer buffer for 10 min, soak the PVDF membrane with methanol for 5 min, and transfer it to the transfer buffer for 10 min after it is fully wetted. Installation The order of transfer clips: black plate, sponge, 3 layers of filter paper, gel, membrane, 3 layers of filter paper, sponge, white plate. Expel bubbles at each step and place the membrane in one go. Transfer the membrane at 300 mA constant current for 100 min in an ice bath.

(5) Blocking: Block with 5% skim milk powder on a shaker at room temperature for 1 h. Rinse with TBST for 10 min×3 times.

(6) Primary antibody incubation: The membrane in contact with the gel should always face up during antibody incubation. Incubate overnight at 4° C. on a shaker. Rinse with TBST the next day.

(7) Secondary antibody incubation: Incubate at room temperature for 1 h. Rinse with TBST.

(8) Luminescence: Use the Kangwei Century Chemiluminescence Detection Kit and scan the detection results with a chemiluminescence imager.

(9) Use Photoshop software to perform semi-quantitative grayscale analysis of the image and calculate the relative expression of the protein.

### 2 Experimental Results

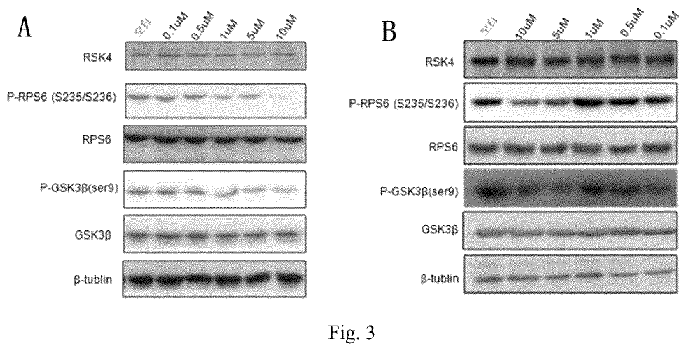

As shown in FIG. 3, different concentrations of Selonsertib were used to stimulate esophageal squamous cell carcinoma cell lines. Western blot analysis revealed that Selonsertib could inhibit phosphorylation of RSK4 downstream substrates and downregulate the expression of P-RPS6 (S235/S236) and P-GSK3β (ser9).

### Example 3: Effect of Selonsertib on Esophageal Squamous Cell Carcinoma Cells

### 1 Experimental Method

This experiment used different concentrations of Selonsertib to stimulate esophageal squamous cell carcinoma cell lines (TE10, ECA109), CCK8 to detect the effect of Selonsertib on esophageal squamous cell carcinoma cell proliferation, and Transwell to detect the effect of Selonsertib on esophageal squamous cell carcinoma cell migration. At the same time, BI-D1870 was used as a positive control, and no drug was used as a blank control (Black).

### 1.1 CCK8 Detection Steps:

The CCK-8 cell proliferation kit of Shanghai Taoshu Biotechnology Co., Ltd. was used.
(1) Cell suspension was inoculated in a 96-well plate, 100 μL per well, 2,000 cells per well.
(2) Culture or give drugs according to experimental needs, and treat for an appropriate time.
(3) Add 10 μL of CCK-8 solution to each well and incubate at 37° C.
(4) The absorbance value was determined by selecting a wavelength of 450 nm on the microplate reader. The growth curve was drawn based on time and absorbance value.

### 1.2 Transwell Migration Assay Steps:

(1) Digest cells. Wash once with PBS, resuspend in serum-free medium, and adjust the cell density to 5×10⁵/ml.
(2) Add 500 μl of culture medium containing 10% fetal bovine serum to the lower chamber of the 24-well plate. Take 200 μl of cell suspension and add it to the chamber. Note that there are no bubbles between the chamber and the lower culture medium.
(3) Culture routinely for 16-24 h.
(4) Aspirate the culture medium in the chamber, fix the cells in the chamber with anhydrous methanol for 15 min, and rinse with PBS.
(5) Color: Add Gumsa staining solution to the 24-well plate, immerse the membrane in the staining solution for 20 min, and wash with PBS.
(6) Carefully wipe off the cells on the inner layer of the membrane with a cotton swab and air-dry the membrane.
(7) Take pictures under a microscope, count 5 high-power fields, and take the average value.

### 2 Experimental Results

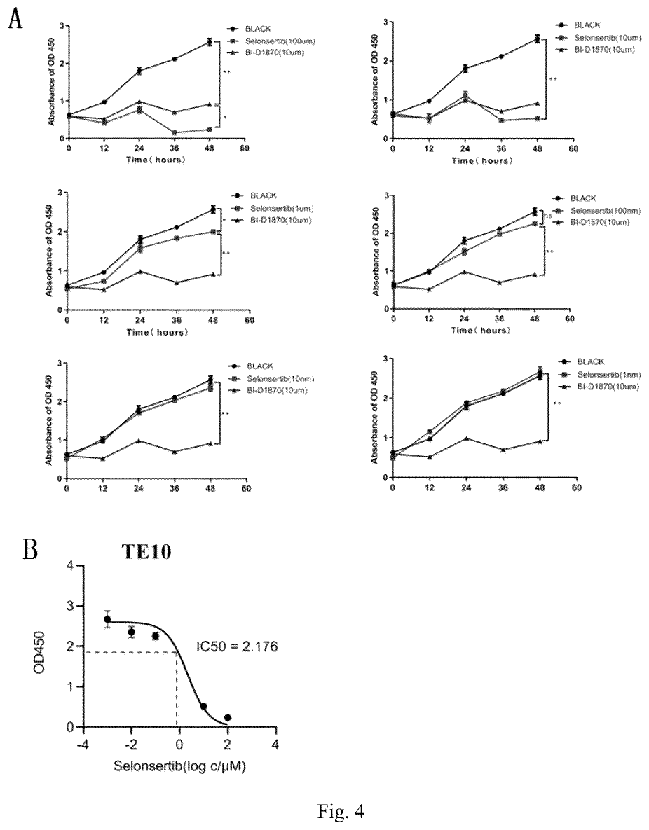

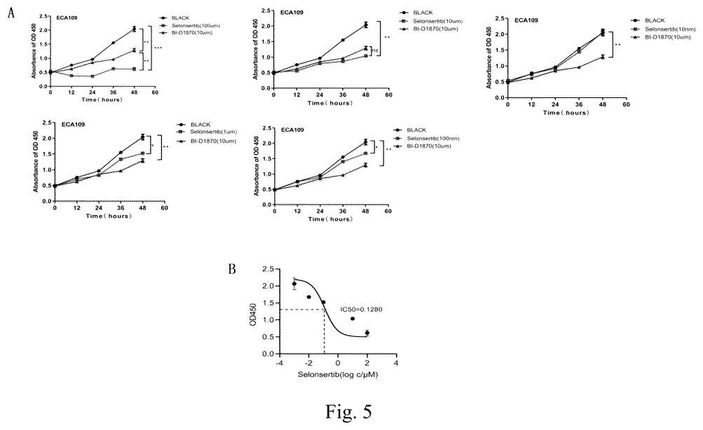

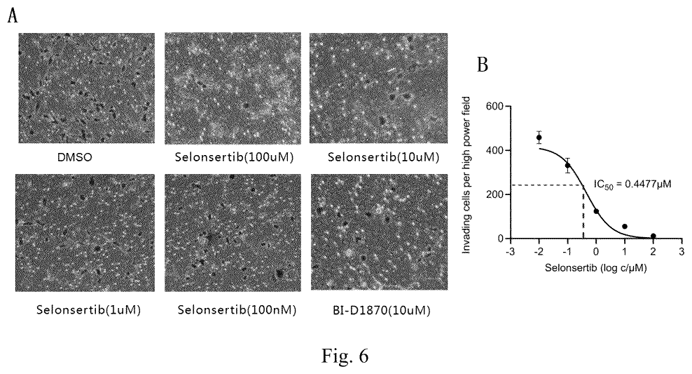

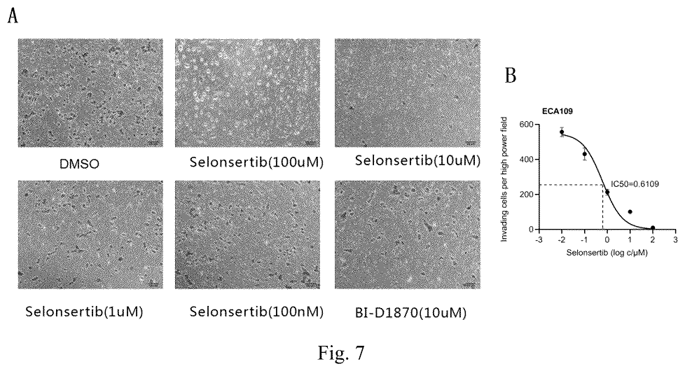

As shown in FIGS. 4-7, compared with the blank control, Selonsertib and BI-D1870 can inhibit the proliferation and migration of TE10 and ECA109, and at the same drug concentration, Selonsertib has a stronger inhibitory effect.

### Example 4: Selonsertib Inhibits the Growth of Esophageal Squamous Cell Carcinoma In Vivo

### 1 Experimental Methods

### Nude Mouse Tumor Formation Experiment:

(1) Nude mouse tumor formation experiment was performed on 4-6 week old female BALB/c nude mice, with 5 nude mice in each group. Each mouse was subcutaneously injected with about 1×106 ESCC cells dissolved in 200 μl sterile PBS.
(2) When the tumor volume reached 150 mm³, the mice were divided into groups and treated differently. Selonsertib was administered intraperitoneally at 15 mg/kg or 30 mg/kg per day (drug dissolution concentration: 5 mg/ml, solvent: 2% DMSO+2% Tween-80+30% PEG300+sterile water), and BI-D1870 was used as a positive control.
(3) The nude mouse tumor formation experiment was observed for 20 days. If the mice were infected or in poor condition, they were treated in time. During this period, the tumor volume was measured every 3-5 days, and the volume calculation formula was length×width 2×0.5.
(4) At the end of the experiment, the mice were killed, the subcutaneous tumors were removed, photographed, weighed, and counted.

### 2 Experimental Results

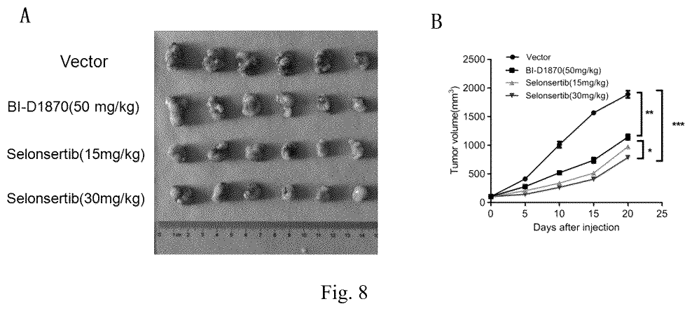

As shown in FIG. 8, compared with the positive control, Selonsertib has a stronger inhibitory effect on tumor growth at a lower dose.

### Example 5: Effect of Selonsertib on EGFR-TKI Targeted Drug-Resistant Strains of Esophageal Squamous Cell Carcinoma

### 1 Experimental Method

The expression level of EGFR and the sensitivity to icotinib and afatinib in various ESCC cell lines (T1, T10, T11, KYSE150, KYSE450, ECA109, EC9706) were detected. The cell line KYSE450 with high EGFR expression and sensitivity to afatinib (low IC50) was selected. The icotinib and afatinib drug concentration increasing mode of action was used to induce KYSE450 cells to produce drug resistance, and a stable cell line resistant to icotinib and afatinib was obtained. The optimal drug concentration gradient for inducing afatinib obtained from the literature reports and the preliminary experiments before formal induction culture was: 100 nM, 300 nM, 500 nM, 800 nM, 1 μM and 5 μM. The initial induction dose of this experiment was 100 nM, and the highest induction dose was 5 μM. During the induction period, the culture medium containing icotinib and afatinib was replaced every 2 days. When the initial induction dose was induced for two weeks and the cell growth was stable, the drug induction dose began to be doubled, and each dose was maintained for 14 days before reaching the highest induction dose. The drug-resistant cells obtained after 10 months of induction culture were named KYSE450/IR KYSE450/AR cell lines.

Selonsertib (2 μm), icotinib (2 μm) or Selonsertib+icotinib (2 μm, the mass ratio of the two is 1:1) was used to stimulate the icotinib-resistant strain of esophageal squamous cell carcinoma (KYSE450-IR), DMSO was used as a blank control, and CCK8 was used to detect (the method is the same as in Example 3) the effects of different drugs on the proliferation of KYSE450-IR.

Selonsertib (2 μm), afatinib (2 μm) or Selonsertib+afatinib (2 μm, the mass ratio of the two is 1:1) was used to stimulate the afatinib-resistant strain of esophageal squamous cell carcinoma (KYSE450/AR), DMSO was used as a blank control, and CCK8 was used to detect (the method is the same as in Example 3) the effects of different drugs on the proliferation of KYSE450/AR.

### 2 Experimental Results

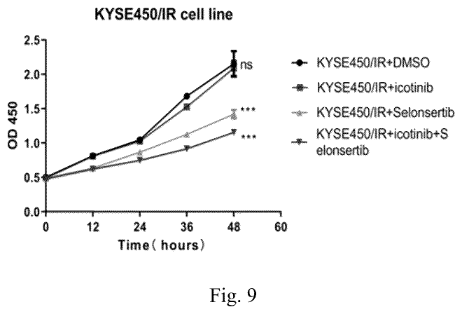

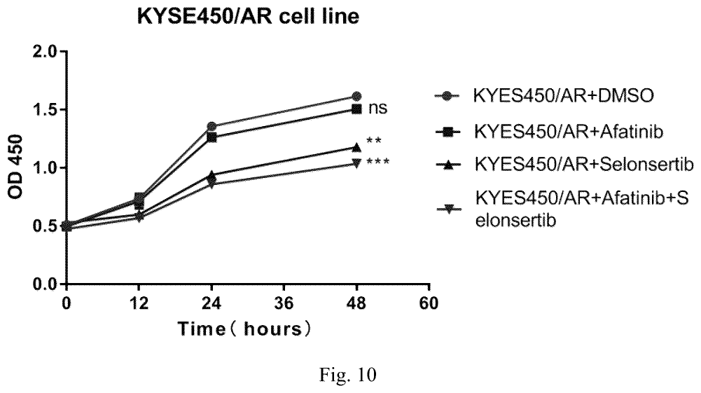

As shown in FIG. 9, Selonsertib or icotinib alone can inhibit the proliferation of KYSE450-IR, but the inhibitory effect of icotinib is extremely low, indicating that KYSE450-IR is resistant to icotinib. When Selonsertib and icotinib are used in combination, the inhibitory effect on KYSE450-IR is significantly stronger than that of Selonsertib alone, indicating that Selonsertib and icotinib can play a synergistic role. The results of KYSE450/AR proliferation are basically consistent with those of KYSE450-IR (FIG. 10), indicating that Selonsertib can reduce the resistance of esophageal squamous cell carcinoma cells to EGFR-TKI targeted drugs. This result provides a new idea for the treatment of esophageal squamous cell carcinoma.

### Example 6: Selonsertib Enhances the Therapeutic Effect of Anti-PD1 on Cancer

### 1 Experimental Methods

(1) B16F10 (melanoma) and LLC (Lewis lung cancer) transplant tumor models were constructed in 6-8 week old C57BL/6 mice, with 6 mice in each group. Each mouse was subcutaneously injected with about 1×106 B16F10 cells and 5×106 LLC cells dissolved in 200 μl sterile PBS.
(2) When the tumor volume reached 150 mm³, the mice were divided into groups and treated differently. Selonsertib was administered by intraperitoneal injection at 15 mg/kg per day; Anti-PD1 was administered at 50 μg/kg once every two days; BI-D1870 was used as a positive control. The Anti-PD1 used in this experiment was specifically In VivoPlus anti-mouse PD-1 (CD279).
(3) The experimental observation time lasted for 10 days (melanoma) or 20 days (Lewis lung cancer). If the mice were infected or in poor condition, they were treated in time. During the period, the tumor volume was measured every 2 days, and the volume calculation formula was length×width 2×0.5.
(4) At the end of the experiment, the mice were killed, the subcutaneous tumors were removed, photographed, weighed, and counted.

### 2 Experimental Results

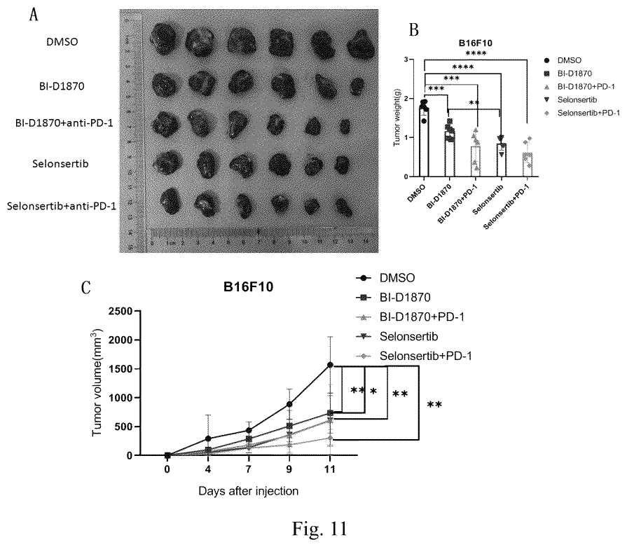

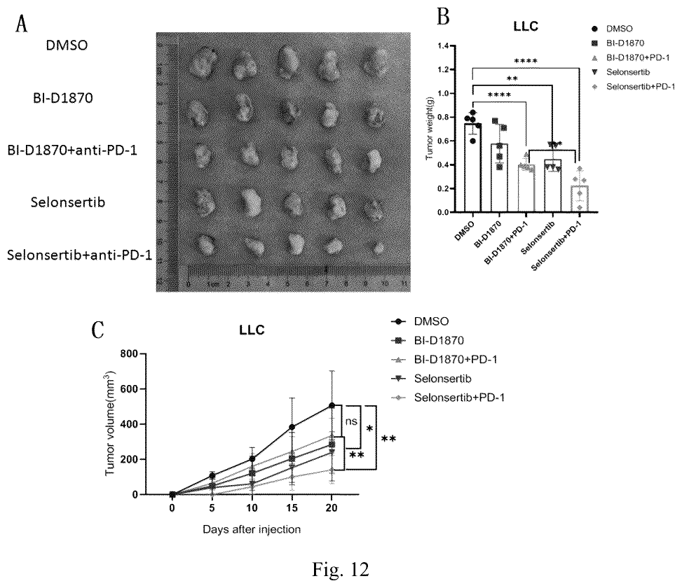

As shown in FIGS. 11 and 12, Selonsertib alone or Selonsertib combined with Anti-PD1 can inhibit the proliferation of melanoma and Lewis lung cancer. The combined use of Selonsertib and Anti-PD1 has a more obvious inhibitory effect on tumor growth.

Although the preferred embodiments of the present invention have been described, those skilled in the art may make additional changes and modifications to these embodiments once they know the basic creative concept. Therefore, the attached claims are intended to be interpreted as including the preferred embodiments and all changes and modifications that fall within the scope of the present invention.

Obviously, those skilled in the art can make various changes and modifications to the present invention without departing from the spirit and scope of the present invention. Thus, if these modifications and variations of the present invention fall within the scope of the claims of the present invention and their equivalents, the present invention is also intended to include these modifications and variations.

1. The use of compound 5-(4-cyclopropyl-1H-imidazol-1-yl)-2-fluoro-N-(6-(4-isopropyl-4H-1,2,4-triazol-3-yl)pyridin-2-yl)-4-methylbenzamide in the preparation of drugs for the treatment of esophageal squamous cell carcinoma is characterized by the fact that the compound 5-(4-cyclopropyl-1H-imidazol-1-yl)-2-fluoro-N-(6-(4-isopropyl-4H-1,2,4-triazol-3-yl) The chemical formula of pyridine-2-yl)-4-methylbenzamide is C24H24FN7O and the structural formula is as follows:

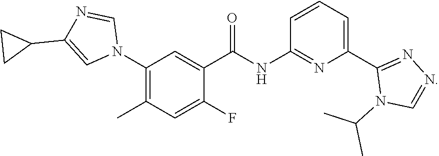 [SMILES: Cc1cc(F)c(C(=O)Nc2cccc(-c3nncn3C(C)C)n2)cc1-n1cnc(C2CC2)c1]

2. The use of claim 1, wherein the compound 5-(4-cyclopropyl-1H-imidazol-1-yl)-2-fluoro-N-(6-(4-isopropyl-4H-1,2,4-triazol-3-yl)pyridin-2-yl)-4-methylbenzamide combined with EGFR-TKI targeted drug is used to prepare drugs for the treatment of esophageal squamous cell carcinoma, The EGFR-TKI is an epidermal growth factor receptor tyrosine kinase inhibitor.

3. The use of claim 2, wherein the EGFR-TKI-targeted drug and compound 5-(4-cyclopropyl-1H-imidazol-1-yl)-2-fluoro-N-(6-(4-isopropyl-4H-1,2,4-triazol-3-yl)pyridine-2-yl)-4-methylbenzamide are administered continuously at the same time or in any sequence.

4. The use of claim 3, wherein the drug is used for the treatment of esophageal squamous cell carcinoma that have developed resistance to EGFR-TKI-targeted drugs, or for the prevention and treatment of esophageal squamous cell carcinoma acquired resistance to EGFR-TKI-targeted drugs, or for the prevention and treatment of esophageal squamous cell carcinoma recur after discontinuation of treatment with EGFR-TKI-targeted drugs; the EGFR-TKI-targeted drugs comprises Icotinib or Afatinib.

5. The use of compound 5-(4-cyclopropyl-1H-imidazol-1-yl)-2-fluoro-N-(6-(4-isopropyl-4H-1,2,4-triazol-3-yl)pyridin-2-yl)-4-methylbenzamide as the only active ingredient in the preparation of drugs for the treatment of melanoma is characterized by the fact that the compound 5-(4-cyclopropyl-1H-imidazol-1-yl)-2-fluoro-N-(6-(4-isopropyl-4H-1,2,4-triazol-3-yl) The chemical formula of pyridine-2-yl)-4-methylbenzamide is C24H24FN7O and the structural formula is as follows:

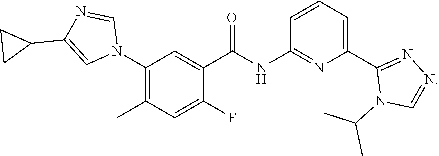 [SMILES: Cc1cc(F)c(C(=O)Nc2cccc(-c3nncn3C(C)C)n2)cc1-n1cnc(C2CC2)c1]

6. The use of claim 5, wherein the compound 5-(4-cyclopropyl-1H-imidazol-1-yl)-2-fluoro-N-(6-(4-isopropyl-4H-1,2,4-triazol-3-yl)pyridine-2-yl)-4-methylbenzamide combined with Anti-PD1 is used, and the Anti-PD1 is a humanization IgG4 antibody is an inhibitor of PD-1, the PD-1 is programmed death receptor 1, and Anti-PD1 includes: Pembrolizumab, Nivolumab, Sintilimab, Toripalimab, Camrelizumab.

7. The use of claim 6, wherein the Anti-PD1 and the compound 5-(4-cyclopropyl-1H-imidazol-1-yl)-2-fluoro-N-(6-(4-isopropyl-4H-1,2,4-triazol-3-yl)pyridine-2-yl)-4-methylbenzamide are administered continuously at the same time or in any sequence.
